(19) United States
(12) Patent Application Publication
(10) Pub. No.: US 2020/0002759 A1
(43) Pub. Date: Jan. 2, 2020

(54) METHODS FOR STUDYING NUCLEOTIDE ACCESSIBILITY IN DNA AND RNA BASED ON LOW-YIELD BISULFITE CONVERSION AND NEXT-GENERATION SEQUENCING

(71) Applicant: William Marsh Rice University, Houston, TX (US)

(72) Inventors: David Yu ZHANG, Houston, TX (US); Jin-Hyung BAE, Houston, TX (US)

(73) Assignee: William Marsh Rice University, Houston, TX (US)

(21) Appl. No.: 16/457,009

(22) Filed: Jun. 28, 2019

Related U.S. Application Data
(60) Provisional application No. 62/691,848, filed on Jun. 29, 2018.

(51) Int. Cl. C12Q 1/6869; C07H 21/02; C07H 21/04; C07H 1/00; C12N 15/10

(52) U.S. Cl. CPC C12Q 1/6869; C07H 21/02; C12N 15/1072; C07H 1/00; C12N 15/1068; C07H 21/04

(57) ABSTRACT

Provided are methods for characterizing nucleotide accessibility and nucleic acid structure at single nucleotide resolution, using a combination of low-yield bisulfite conversion and next-generation sequencing (NGS). Cytosine (C) nucleotides that are in base-paired states or bound to proteins, etc. are less accessible to chemical reactions and thus exhibit lower bisulfite conversion yields. Analysis of NGS results of a low-yield bisulfite conversion product can thus inform nucleotide accessibility and nucleic acid structure. Compared to other methods for chemical probing of nucleic acid structure, the present methods provide higher information throughput, because each NGS read simultaneously reports on the base pair status of multiple nucleotides.

### REFERENCE TO RELATED APPLICATIONS

The present application claims the priority benefit of U.S. provisional application No. 62/691,848, filed Jun. 29, 2018, the entire contents of which is incorporated herein by reference.

### STATEMENT REGARDING FEDERALLY SPONSORED RESEARCH

This invention was made with government support under Grant No. RO1 HG008752 awarded by the National Institutes of Health. The government has certain rights in the invention.

### REFERENCE TO A SEQUENCE LISTING

The instant application contains a Sequence Listing, which has been submitted in ASCII format via EFS-Web and is hereby incorporated by reference in its entirety. Said ASCII copy, created on Jun. 26, 2019, is named RICEP0050US_ST25.txt and is 706 kilobytes in size.

### BACKGROUND

### 1. Field

The present invention relates generally to the field of molecular biology. More particularly, it concerns methods of low-yield bisulfite conversion of DNA and RNA.

### 2. Description of Related Art

Nucleic acids such as DNA and RNA can serve both as therapeutic drugs (e.g., RNAi therapy and mRNA therapy) and as drug targets (e.g., ribosomal RNA in bacteria for antibiotics). Thus, understanding the structure of nucleic acids and the accessibility of different nucleotides in a nucleic acid to biochemical reactions is of value to the pharmaceutical industry. Accurate analysis of nucleic acid structure before and after introduction of a potential protein or small molecule drug can confirm the effectiveness of the lead, and further verify the mechanism of drug action. Additionally, many biological pathways remain to date imperfectly elucidated; thus, understanding the interactions between nucleic acids and proteins is of great interest to the scientific community.

Over the past decades, X-ray crystallography (XRC) has been used to produce gold-standard structures of nucleic acids, but its low throughput, high sample requirement, and operation difficulty means that XRC cannot fulfill the current demand for high-throughput analysis of many different DNA and RNA molecules. Next-generation sequencing (NGS) is a powerful way of simultaneously analyzing millions of different nucleic acid molecules, and can be potentially combined with chemical probing methods such as SHAPE (Selective 2′ Hydroxyl Acylation analyzed by Primer Extension) (Lucks et al., 2011) to study the chemical accessibility of different nucleotides in a DNA molecule. The chemical accessibility is then correlated to nucleotide base pair status, with nucleotides more frequently paired generally being less accessible to chemical reactions.

Simultaneously, bisulfite ions at high concentrations are known to selectively react with the cytosine (C) nucleotides, converting them to uracil (U) nucleotides. In contrast, cytosines that are methylated are protected from this bisulfite conversion. Consequently, researchers have over the years turned bisulfite conversion into a workhorse for the study of epigenetics, differentiating hypermethylated vs. hypomethylated promoters. For this use of bisulfite conversion, users typically drive the bisulfite conversion reaction to completion through high temperatures, high bisulfite concentrations, and long reaction times. Driving the conversion to completion prevents ambiguities on whether a particular site is a methylated C or if it is an unmethylated C that failed to convert.

### SUMMARY

Provided herein are methods of bisulfite conversion, in which the conversion efficiency is intentionally designed to be low, so that differences in nucleotide accessibility (e.g., from being in a base-paired or protein-bound state) are reflected in bisulfite conversion yields.

In one embodiment, provided herein are methods for low-yield conversion of unmethylated cytosine nucleotides to uracil (U) nucleotides in a target nucleic acid molecule, the method comprising: (a) introducing a bisulfite solution to a sample comprising the target nucleic acid molecule to achieve a final bisulfite concentration of between 0.1 M and 10 M; (b) allowing the bisulfite conversion to react a temperature between 4° C. and 70° C.; (c) stopping the bisulfite conversion reaction through removal of the excess bisulfite; and (d) performing desulfonation.

In some aspects, unmethylated cytosines that do not participate in base pairing have a C to U conversion rate of no more than 95%, no more than 90%, no more than 85%, no more than 80%, no more than 75%, no more than 70%, no more than 65%, no more than 60%, no more than 55%, or no more than 50%.

In some aspects, the bisulfite concentration is between 0.1 M and 10 M, 0.5 M and 10 M, 0.1 and 5 M, 0.5 and 5 M, 4 M and 10 M, 4 M and 5 M, 0.1 M and 1 M, 0.5 M and 1 M, or any range derivable therein. In some aspects, the bisulfite concentration may be about 0.1 M, 0.5 M, 1 M, 1.5 M, 2 M, 2.5 M, 3 M, 3.5 M, 4 M, 4.5 M, 5 M, 5.5 M, 6 M, 6.5 M, 7 M, 7.5 M, 8 M, 8.5 M, 9 M, 9.5 M, or 10 M.

In some aspects, the reaction proceeds at a temperature between 4° C. and 70° C., 4° C. and 55° C., 37° C. and 70° C., 37° C. and 55° C., 45° C. and 70° C., 45° C. and 55° C., or any range derivable therein. In some aspects, the reaction proceeds at a temperature of about 4° C., 5° C., 10° C., 15° C., 20° C., 25° C., 30° C., 35° C., 40° C., 45° C., 50° C., 55° C., 60° C., 65° C., or 70° C.

In some aspects, the reaction proceeds for a time of between 1 minutes and 60 minutes, 10 seconds and 20 minutes, 5 minutes and 60 minutes, 30 minutes and 12 hours, or any range derivable therein. In some aspects, the reaction proceeds for a time of about 10 seconds, 30 seconds, 1 minute, 5 minutes, 10 minutes, 15 minutes, 20 minutes, 25 minutes, 30 minute, 35 minutes, 40 minutes, 45 minutes, 50 minutes, 55 minutes, 60 minutes, 65 minutes, 70 minutes, 75 minutes, 80 minutes, 85 minutes, 90 minutes, 2 hours, 3 hours, 4 hours, 5 hours, 6 hours, 7 hours, 8 hours, 9 hours, 10 hours, 11 hours, or 12 hours.

In some aspects, the bisulfite concentration is between 4 M and 10 M, and the reaction proceeds at a temperature between 4° C. and 55° C. for between 1 minutes and 60 minutes. In some aspects, the bisulfite concentration is between 4 M and 10 M, and the reaction proceeds at a temperature between 45° C. and 70° C. for between 10 seconds and 20 minutes. In some aspects, the bisulfite concentration is between 0.5 M and 5 M, and the reaction proceeds at a temperature between 45° C. and 70° C. for between 5 minutes and 60 minutes. In some aspects, the bisulfite concentration is between 0.1 M and 1 M, and the reaction proceeds at a temperature between 37° C. and 70° C. for between 30 minutes and 12 hours.

In some aspects, the target nucleic acid molecule is DNA, including but not limited in origin to genomic DNA from a cell or tissue sample, viral DNA, cell-free DNA, or synthetic DNA. In some aspects, the target nucleic acid molecule is RNA, including but not limited in origin to mRNA or non-coding RNA from a cell or tissue sample, viral RNA, cell-free RNA, or synthetic RNA.

In some aspects, the bisulfite conversion is stopped by separating the bisulfite from the target nucleic acid. In some aspects, the bisulfite is removed by a buffer exchange method, including but not limited to column purification or solid phase reversible mobilization (SPRI).

In some aspects, desulfonation is performed at a pH over 12.

In one embodiment, provided herein are methods of determining a nucleotide accessibility status of cytosine (C) nucleotides in a target nucleic acid molecule, the method comprising: (a) performing a low-yield bisulfite conversion reaction to convert a fraction of cytosine nucleotides in a population of the target nucleic acid molecules to uracil (U) nucleotides; (b) analyzing the converted nucleic acid molecules at each nucleotide position originally known to be a cytosine, to determine or estimate a conversion fraction of all molecules of the population that is converted at each nucleotide position originally known to be a cytosine; and (c) mapping the conversion fraction to a nucleotide accessibility status.

In some aspects, analyzing comprises sequencing the converted nucleic acid molecules. In some aspects, sequencing comprises massively parallel sequencing-by-synthesis, also known as next-generation sequencing (NGS). In some aspects, sequencing is sequencing via translocation current across a nanopore.

In some aspects, the fraction of all molecules of a target nucleic acid that are read as thymine (T) or uracil (U) at a given nucleotide is computed or estimated from sequencing data.

In some aspects, the low-yield bisulfite conversion converts unpaired cytosine nucleotides in a nucleic acid molecule with an efficacy of no more than 95%, no more than 90%, no more than 85%, no more than 80%, no more than 75%, no more than 70%, no more than 65%, no more than 60%, no more than 55%, or no more than 50%.

In some aspects, low-yield bisulfite conversion is achieved through (1) bisulfite concentrations of less than 4 M, (2) reaction with bisulfite solution for less than 60 minutes, and/or (3) reaction with bisulfite solution at a temperature less than 70° C.

In some aspects, mapping the conversion fraction to nucleotide accessibility status comprises comparing the conversion fraction at a C nucleotide position to conversion fractions of all other C nucleotide positions in the same nucleic acid species. In some aspects, mapping the conversion fraction to nucleotide accessibility status comprises comparing the conversion fraction at a C nucleotide position to conversion fractions of all other C nucleotide positions in the other all nucleic acid species within the same multiplexed conversion reaction. In some aspects, mapping the conversion fraction to nucleotide accessibility status comprises comparing the conversion fraction at a C nucleotide position to an information database of conversion fractions of C nucleotides in different accessibility states, under the same low-yield bisulfite conversion reaction conditions.

In some aspects, the nucleotide accessibility status is used to determine the base pair status of different nucleotides, in order to inform the correctness of a proposed secondary and/or tertiary structure of the nucleic acid. In some aspects, the nucleotide accessibility status is used to determine the nucleotide positions where a small molecule or protein is bound to the nucleic acid. In certain aspects, the small molecule or protein is a therapeutic drug and the nucleic acid is an endogenous RNA drug target. In certain aspects, the protein is an endogenous drug target and the nucleic acid is a therapeutic drug.

In one embodiment, provided herein are methods of determining a nucleotide accessibility status of cytosine (C) nucleotides in a target nucleic acid molecule, the method comprising: (a) performing a low-yield bisulfite conversion reaction to convert a fraction of cytosine nucleotides in a first sample of a population of the target nucleic acid molecules to uracil (U) nucleotides for a defined amount of time t₁; (b) constructing a next-generation sequencing (NGS) library based on the converted nucleic acid molecules and running NGS; (c) analyzing the NGS reads to determine, for each nucleotide position originally known to be a cytosine, the fraction of all molecules of the converted nucleic acid molecules that is converted to determine a bisulfite conversion rate; (d) repeating steps (a) through (c) for a second sample of the same population of nucleic acid molecules, subject to a low-yield bisulfite conversion reaction for a different amount of time t₂; and (e) determining the accessibility of a nucleotide in the target nucleic acid molecules based on the combined analysis of the bisulfite conversion rates from both time points.

In some aspects, the low-yield bisulfite conversion converts unpaired cytosine nucleotides in a nucleic acid molecule with an efficacy of no more than 95%, no more than 90%, no more than 85%, no more than 80%, no more than 75%, no more than 70%, no more than 65%, no more than 60%, no more than 55%, or no more than 50%.

In some aspects, low-yield bisulfite conversion is achieved through (1) bisulfite concentrations of less than 4 M, (2) reaction with bisulfite solution for less than 60 minutes, or (3) reaction with bisulfite solution at a temperature less than 70° C.

In some aspects, mapping the conversion fraction to nucleotide accessibility status comprises comparing the conversion fraction at a C nucleotide position to conversion fractions of all other C nucleotide positions in the same nucleic acid species. In some aspects, mapping the conversion fraction to nucleotide accessibility status comprises comparing the conversion fraction at a C nucleotide position to conversion fractions of all other C nucleotide positions in the other all nucleic acid species within the same multiplexed conversion reaction. In some aspects, mapping the conversion fraction to nucleotide accessibility status comprises comparing the conversion fraction at a C nucleotide position to an information database of conversion fractions of C nucleotides in different accessibility states, under the same low-yield bisulfite conversion reaction conditions.

In some aspects, the nucleotide accessibility status is used to determine the base pair status of different nucleotides, in order to inform the correctness of a proposed secondary and/or tertiary structure of the nucleic acid. In some aspects, the nucleotide accessibility status is used to determine the nucleotide positions where a small molecule or protein is bound to the nucleic acid. In certain aspects, the small molecule or protein is a therapeutic drug, and the nucleic acid is an endogenous RNA drug target. In certain aspects, the protein is an endogenous drug target, and the nucleic acid is a therapeutic drug.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Workflow for low-yield bisulfite conversion and NGS library preparation. The sodium bisulfite reaction was performed for a significantly shorter amount of time and at a lower temperature than recommended by the kit provider (Zymo Research), in order to accentuate the difference in C>U conversion rates between paired and unpaired nucleotides. The target-specific halves of the splint oligos (shown in black) contain degenerate purine nucleotides R, corresponding to a mixture of G and A, in order to efficiently bind all possible conversion products.

FIG. 2: Control oligo demonstrating validity of low-yield bisulfite conversion as a method of probing secondary structure. This oligo (SEQ ID NO: 9) was rationally designed to have a very stable and unambiguous minimum free energy (MFE) structure containing a large hairpin. The observed conversion rate for each C nucleotide, based on NGS experiments, is listed in the illustration. As expected, all C nucleotides predicted to be unpaired (green) exhibit high conversion rates, and all C nucleotides predicted to be paired (red) exhibit low conversion rates. The C nucleotides near the ends of the predicted duplex region (yellow) show intermediate conversion rates, due to base breathing that reduce the likelihood of the nucleotide being paired at equilibrium.

FIG. 3: Controlling bisulfite conversion yield via reaction time. Here, the temperature of the reaction was set to 37° C. to mimic biological conditions, and different reaction times resulted in different C>U conversion rates for the different nucleotides. Importantly, this time series shows a graded response of conversion rate of C nucleotides in base-paired states, with lower conversion in C nucleotides “deeper” in the hairpin stem.

FIGS. 4A-C: Use of low-yield bisulfite conversion and NGS results to inform nucleic acid structure. (FIG. 4A) Minimum free energy (MFE) structure of an STK11 exon subsequence as predicted by standard thermodynamic parameters versus using updated TEEM parameters for bulges and mismatches. The nucleotide bases are color-coded by their accessibility in the MFE structure predicted by standard parameters. Even though the two MFE structures have similar folding energies, the structure and base accessibility are very different. (FIG. 4B) Two different secondary structures of the target nucleic acid, a subsequence of the human SMARCA4 gene, are proposed based on different thermodynamics parameters. Nucleotides here are colored/shaded based on the predicted state using thermodynamics parameters from SantaLucia, 1998 (left). The right structure is predicted based on forthcoming thermodynamic parameters from Bae and Zhang, 2018. (FIG. 4C) Predicted MFE structures of a KEAP1 exon subsequence based on updated TEEM parameters, colored by predicted structure using standard parameters.

FIGS. 5A-B: Chemical assay of DNA secondary structure using low-yield bisulfite conversion and next-generation sequencing (NGS). (FIG. 5A) Low-yield bisulfite conversion rates of C nucleotides in the 3 oligos shown in FIGS. 4A-C. Boxes indicate sample standard deviations and horizontal lines show mean values. The nucleotides predicted to be paired by TEEM parameters (PP) are statistically significantly lower in conversion rate than the nucleotides predicted to be unpaired (PU); the left panel shows the aggregated data from all 3 oligos. P-values shown are based on Mann-Whitney tests. (FIG. 5B) Predicted paired (PP) nucleotides and predicted unpaired nucleotides (PU), based on SantaLucia 2004 parameters, do not show statistically significant differences in bisulfite conversion rates. P-values shown are based on Mann-Whitney tests.

FIG. 6: NGS workflow starting from a pool of 1,967 oligonucleotides, which were all 150 nucleotides long. The first step was PCR where one of the primers had dU at 3′-end and three phosphorothioate bonds at 5′-end. Resulting amplicons had both USER and BciVI restriction sites. After using BciVI, Lambda exonuclease, and USER, primer binding regions were removed, and only the 100-nucleotide long areas of interest remained. Low-yield bisulfite conversion was then performed, followed by two ligation steps to attach NGS adaptors with the help of random hexamers on the guiding oligos. NGS library preparation was finished by running index PCR.

FIG. 7: Data processing. An NGS library generated 5.3 million of raw reads with some of C converted to T as a result of Low Yield Bisulfite Conversion. For alignment purpose, all C and T were written as Y temporarily in both the raw reads and the reference sequences. After selecting only perfectly aligned reads, 2.6 million reads were left with the median read depth of 603. All C and T were then recovered from Y. To make data processing faster and more efficient, C converted to T during Low Yield Bisulfite was treated as 1 whereas C unconverted to T was treated as 0. Finding the dominant structure of an oligo was done by using average conversion of each C, and finding subpopulations of structures for each oligo was done by clustering the reads instead of calculating the average conversion.

FIG. 8: Conversion differences among groups. Motifs of a secondary structure were classified into 3 groups: Flank, Stem, and Loop. When mean conversions of each C were grouped according to where the C is located, there are clear differences among three groups.

FIG. 9: Loop pattern 1. Mean conversions at Loop were grouped by the length of Loop when the length of Stem was always 20 nucleotides.

FIG. 10: Loop pattern 2. Mean conversion at Loop were plotted against the free energy of hybridization of their own Stem.

FIG. 11: Subpopulations. Conversions of an oligonucleotide that was designed to have two competing structures in equilibrium. All reads of an oligonucleotide were visualized by displaying converted C in white and unconverted C in black. Each row shows all C in one read, and each column shows specific position of C. The reads were sorted by conversions at 8^{th} and 9^{th} C.

### DETAILED DESCRIPTION

Provided herein are methods of bisulfite conversion, in which the conversion efficiency is intentionally designed to be low, so that differences in nucleotide accessibility (e.g., from being in a base-paired or protein-bound state) are reflected in bisulfite conversion yields.

Compared to prior bisulfite conversion methods, this disclosure intentionally uses lower bisulfite concentrations, shorter reaction times, and/or lower temperatures to reduce the yield of conversion. In particular, the goal of low bisulfite conversion yield is uniquely compatible with the goal of understanding native structures of nucleic acids at physiological conditions such as 37° C. and intracellular salinity conditions. These methods systematically use bisulfite conversion to characterize nucleotide accessibility, and the first reported method of analyzing low-yield bisulfite conversion products using NGS. Additionally, low-yield bisulfite conversion can be applied to the study of nucleotide accessibility for both DNA and RNA molecules. Because the majority of nucleic acid drug targets are mRNA molecules, because the majority of nucleic acid drugs are siRNAs or mRNAs, and because RNA has relatively fewer methylated cytosines that can obfuscate interpretation of conversion rates, low-yield bisulfite conversion may be well-suited for profiling RNA nucleotide accessibility to inform therapeutics development and characterization.

Compared to prior chemical probing methods for querying nucleotide accessibility such as SHAPE-Seq (Lucks et al., 2011), which effectively causes nucleic acid breaks preferentially at accessible nucleotides, this disclosure provides significantly higher throughput of information, because each NGS read simultaneously contributes information on the accessibility of multiple different cytosine nucleotides and furthermore does not have an information bias towards one end of the nucleic acid molecule. Furthermore, the low-yield bisulfite conversion method allows co-analysis of conversion rates of the same nucleotide under different bisulfite conversion reaction times in order to estimate the “depth” of structure or accessibility.

Applications of the disclosure include uses that help to inform the effectiveness of potential drugs or clarify biological pathways. DNA and RNA can act as both drug targets and as therapeutic molecules, so understanding the nucleotides that participate in different reactions is valuable to drug development. The secondary and tertiary structures of RNA can inform the design of new antibiotics, via information regarding accessibility of different ribosomal RNA targets. The secondary and tertiary structure information is also potentially important for new classes of RNA drugs, including lncRNA drugs. There is a large research use market for understanding secondary structure of RNA for design of in situ hybridization probes. Likewise, understanding the secondary structure of genomic DNA can inform the better design of PCR primers and hybrid-capture probes for genomics research and molecular diagnostics.

### I. BISULFITE CONVERSION

The term “bisulfite-converted DNA” as used herein refers to DNA that has been subjected to sodium bisulfite such that at least some of the unmethylated cytosines in the DNA are converted to uracil.

A typical bisulfite conversion reaction consists of three steps: sulfonation, deamination, and desulfonation. During sulfonation, a sulfonic acid functional group from a bisulfite ion replaces a hydrogen atom in cytosine, which causes spontaneous deamination of 6-sulfo-cytosine. In some embodiments, bisulfite exists in the form of sodium bisulfite. In some embodiments, the concentration of sodium bisulfite is approximately 5.0 M. In other embodiments, the concentration of sodium bisulfite is lower to control the rate of bisulfite conversion. For example, the concentration of sodium bisulfite may be 4.75 M, 4.5 M, 4.25 M, 4 M, 3.75 M, 3.5 M, 3.25 M, 3 M, 2.75 M, 2.5 M, 2.25 M, 2 M, 1.75 M, 1.5 M, 1.25 M, 1 M, 0.75 M, 0.5 M, 0.25 M, or 0.15 M. In some embodiments, pH is adjusted to 5.0 by adding NaOH. In some embodiments, antioxidants, such as hydroquinone, can be added to prevent DNA degradation by radicals. At the end of the desulfonation reaction, cytosine nucleotides are converted with some probability to uracil nucleotides. Cytosine nucleotides that are less accessible, for example due to being base paired or bound to a protein, are converted with a lower yield than more accessible cytosines.

In addition to the bisulfite concentration, the reaction time and temperature also affect the conversion yield. In some embodiments, the conversion is performed at 55° C. for 5 minutes or 20 minutes. In some embodiments, temperature is dropped to 4° C. at the end to slow down sulfonation and deamination before removing bisulfite. Bisulfite concentration can also be controlled to match salinity of interest. In one embodiment, the bisulfite solution was diluted and the conversion was performed at 98° C. for 5 minutes with fragmented Mycobacterium tuberculosis genomic DNA. The resulting conversion products were analyzed by NGS and the average conversion rate of all cytosines were as follows:

| [Bisulfite] | 5.00M | 4.75M | 4.50M | 4.25M | 4.00M | 3.75M | 3.5M |
|---|---|---|---|---|---|---|---|
| Conversion | 63.1% | 55.4% | 46.3% | 36.8% | 25.5% | 14.9% | 7.4% |

The desulfonation step removes the sulfonic acid functional group from 6-sulfo-uracil by increasing pH, resulting in uracil instead of cytosine. In some embodiments, desulfonation is performed by dissolving DNA into basic solution, such as 0.2 M NaOH. In some embodiments, desulfonation is performed at room temperature for 15 minutes.

### II. NEXT-GENERATION SEQUENCING (NGS)

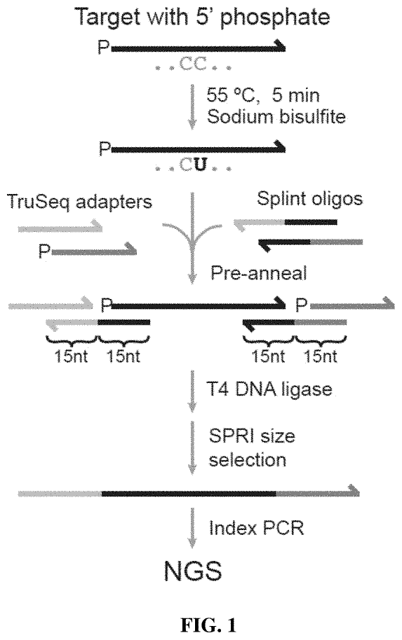

The inventors use NGS to analyze bisulfite conversion yields in a high-throughput manner. The converted nucleic acid products are prepared into an NGS library via ligation or PCR addition of adapter sequences. In some embodiments, Illumina adapter sequences are first attached to target oligos after bisulfite conversion via ligation. In some embodiments, two oligos with the adapter sequences are attached to 5′-end and 3′-end of target by ligation. In some embodiments, the ligation is assisted by splint oligos, which hold a target oligo and two adapter oligos together to exploit the nick repair ability of DNA ligases. In some embodiments, a mixture of a target oligo, two splint oligos, and two adapter oligos are pre-annealed before a ligation. In some embodiments, a ligation is performed with T4 DNA ligase. In other embodiments, a ligation is performed with single-stranded DNA ligase and no splint oligos. In some embodiments, PCR is used to attach adapters and/or index sequences to the ligation product. An example workflow for creating an NGS library from the bisulfite conversion products is shown in FIG. 1. Note that uracil (U) nucleotides are either converted in the NGS library preparation process to thymine (T) nucleotides, or read by the NGS as thymine nucleotides during the bridge PCR or emulsion PCR pre-amplification process.

Any technique for sequencing nucleic acids known to those skilled in the art can be used in the methods of the present disclosure. DNA sequencing techniques include classic dideoxy sequencing reactions (Sanger method) using labeled terminators or primers and gel separation in slab or capillary, sequencing-by-synthesis using reversibly terminated labeled nucleotides, pyrosequencing, 454 sequencing, allele specific hybridization to a library of labeled oligonucleotide probes, sequencing-by-synthesis using allele specific hybridization to a library of labeled clones that is followed by ligation, real time monitoring of the incorporation of labeled nucleotides during a polymerization step, and SOLiD sequencing.

The nucleic acid library may be generated with an approach compatible with Illumina sequencing such as a Nextera™ DNA sample prep kit, and additional approaches for generating Illumina next-generation sequencing library preparation are described, e.g., in Oyola et al. (2012). In other embodiments, a nucleic acid library is generated with a method compatible with a SOLiD™ or Ion Torrent sequencing method (e.g., a SOLiD® Fragment Library Construction Kit, a SOLiD® Mate-Paired Library Construction Kit, SOLiD® ChIP-Seq Kit, a SOLiD® Total RNA-Seq Kit, a SOLiD® SAGE™ Kit, a Ambion® RNA-Seq Library Construction Kit, etc.). Additional methods for next-generation sequencing methods, including various methods for library construction that may be used with embodiments of the present disclosure are described, e.g., in Pareek (2011) and Thudi (2012).

In particular aspects, the sequencing technologies used in the methods of the present disclosure include the HiSeg™ system (e.g., HiSeg™ 2000 and HiSeg™ 1000) and the MiSeg™ system from Illumina, Inc. The HiSeg™ system is based on massively parallel sequencing of millions of fragments using attachment of randomly fragmented genomic DNA to a planar, optically transparent surface and solid phase amplification to create a high-density sequencing flow cell with millions of clusters, each containing about 1,000 copies of template per sq. cm. These templates are sequenced using four-color DNA sequencing-by-synthesis technology. The MiSeg™ system uses TruSeq™, Illumina's reversible terminator-based sequencing-by-synthesis.

Another example of a DNA sequencing platform is the QIAGEN GeneReader platform—a next generation sequencing (NGS) platform utilizing proprietary modified nucleotides whose 3′ OH groups are reversely terminated by a small moiety to perform sequencing-by-synthesis (SBS) in a massively parallel manner. Briefly, the sequencing templates are first clonally amplified on a solid surface (such as beads) to generate hundreds of thousands of identical copies for each individual sequencing template, denaturized to generate single-stranded sequencing templates, hybridized with sequencing primer, and then immobilized on the flow cell. The immobilized sequencing templates are then subjected to a nucleotide incorporation reaction in a reaction mix that includes modified nucleotides with a cleavable 3′ blocking group that enables the incorporation and detection of only one specific nucleotide onto each sequencing template in each cycle. See U.S. Pat. Nos. 6,664,079; 8,612,161; and 8,623,598, each of which is incorporated by reference herein.

Another example of a DNA sequencing platform is the Ion Torrent PGM™ sequencer (Thermo Fisher) and the Ion Torrent Proton™ Sequencer (Thermo Fisher), which are ion-based sequencing systems that sequence nucleic acid templates by detecting ions produced as a byproduct of nucleotide incorporation. Typically, hydrogen ions are released as byproducts of nucleotide incorporations occurring during template-dependent nucleic acid synthesis by a polymerase. The Ion Torrent PGM™ sequencer and Ion Proton™ Sequencer detect the nucleotide incorporations by detecting the hydrogen ion byproducts of the nucleotide incorporations. The Ion Torrent PGM™ sequencer and Ion Torrent Proton™ sequencer include a plurality of nucleic acid templates to be sequenced, each template disposed within a respective sequencing reaction well in an array. The wells of the array are each coupled to at least one ion sensor that can detect the release of H+ ions or changes in solution pH produced as a byproduct of nucleotide incorporation. The ion sensor comprises a field effect transistor (FET) coupled to an ion-sensitive detection layer that can sense the presence of H+ ions or changes in solution pH. The ion sensor provides output signals indicative of nucleotide incorporation, which can be represented as voltage changes whose magnitude correlates with the H+ ion concentration in a respective well or reaction chamber. Different nucleotide types are flowed serially into the reaction chamber, and are incorporated by the polymerase into an extending primer (or polymerization site) in an order determined by the sequence of the template. Each nucleotide incorporation is accompanied by the release of H+ ions in the reaction well, along with a concomitant change in the localized pH. The release of H+ ions is registered by the FET of the sensor, which produces signals indicating the occurrence of the nucleotide incorporation. Nucleotides that are not incorporated during a particular nucleotide flow will not produce signals. The amplitude of the signals from the FET may also be correlated with the number of nucleotides of a particular type incorporated into the extending nucleic acid molecule thereby permitting homopolymer regions to be resolved. Thus, during a run of the sequencer multiple nucleotide flows into the reaction chamber along with incorporation monitoring across a multiplicity of wells or reaction chambers permit the instrument to resolve the sequence of many nucleic acid templates simultaneously. Further details regarding the compositions, design and operation of the Ion Torrent PGM™ sequencer can be found, for example, in U.S. Pat. Publn. Nos. 2009/0026082; 2010/0137143; and 2010/0282617, all of which are incorporated by reference herein in their entireties.

Another example of a DNA sequencing technique that can be used in the methods of the present disclosure is 454 sequencing (Roche) (Margulies et al., 2005). 454 sequencing involves two steps. In the first step, DNA is sheared into fragments of approximately 300-800 base pairs, and the fragments are blunt ended. Oligonucleotide adaptors are then ligated to the ends of the fragments. The adaptors serve as primers for amplification and sequencing of the fragments. The fragments can be attached to DNA capture beads, e.g., streptavidin-coated beads using, e.g., Adaptor B, which contains 5′-biotin tag. The fragments attached to the beads are PCR amplified within droplets of an oil-water emulsion. The result is multiple copies of clonally amplified DNA fragments on each bead. In the second step, the beads are captured in wells (pico-liter sized). Pyrosequencing is performed on each DNA fragment in parallel. Addition of one or more nucleotides generates a light signal that is recorded by a CCD camera in a sequencing instrument. The signal strength is proportional to the number of nucleotides incorporated.

Another example of a DNA sequencing technique that can be used in the methods of the present disclosure is SOLiD technology (Life Technologies, Inc.). In SOLiD sequencing, genomic DNA is sheared into fragments, and adaptors are attached to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations are prepared in microreactors containing beads, primers, template, and PCR components. Following PCR, the templates are denatured and beads are enriched to separate the beads with extended templates. Templates on the selected beads are subjected to a 3′ modification that permits bonding to a glass slide.

Another example of a DNA sequencing technique that can be used in the methods of the present disclosure is the IonTorrent system (Life Technologies, Inc.). Ion Torrent uses a high-density array of micro-machined wells to perform this biochemical process in a massively parallel way. Each well holds a different DNA template. Beneath the wells is an ion-sensitive layer and beneath that a proprietary Ion sensor. If a nucleotide, for example a C, is added to a DNA template and is then incorporated into a strand of DNA, a hydrogen ion will be released. The charge from that ion will change the pH of the solution, which can be detected by the proprietary ion sensor. The sequencer will call the base, going directly from chemical information to digital information. The Ion Personal Genome Machine (PGM™) sequencer then sequentially floods the chip with one nucleotide after another. If the next nucleotide that floods the chip is not a match, no voltage change will be recorded and no base will be called. If there are two identical bases on the DNA strand, the voltage will be double, and the chip will record two identical bases called. Because this is direct detection—no scanning, no cameras, no light—each nucleotide incorporation is recorded in seconds.

Another example of a sequencing technology that can be used in the methods of the present disclosure includes the single molecule, real-time (SMRT™) technology of Pacific Biosciences. In SMRT™, each of the four DNA bases is attached to one of four different fluorescent dyes. These dyes are phospholinked. A single DNA polymerase is immobilized with a single molecule of template single stranded DNA at the bottom of a zero-mode waveguide (ZMW). A ZMW is a confinement structure which enables observation of incorporation of a single nucleotide by DNA polymerase against the background of fluorescent nucleotides that rapidly diffuse in and out of the ZMW (in microseconds). It takes several milliseconds to incorporate a nucleotide into a growing strand. During this time, the fluorescent label is excited and produces a fluorescent signal, and the fluorescent tag is cleaved off. Detection of the corresponding fluorescence of the dye indicates which base was incorporated. The process is repeated.

A further sequencing platform includes the CGA Platform (Complete Genomics). The CGA technology is based on preparation of circular DNA libraries and rolling circle amplification (RCA) to generate DNA nanoballs that are arrayed on a solid support (Drmanac et al. 2010). Complete genomics' CGA Platform uses a novel strategy called combinatorial probe anchor ligation (cPAL) for sequencing. The process begins by hybridization between an anchor molecule and one of the unique adapters. Four degenerate 9-mer oligonucleotides are labeled with specific fluorophores that correspond to a specific nucleotide (A, C, G, or T) in the first position of the probe. Sequence determination occurs in a reaction where the correct matching probe is hybridized to a template and ligated to the anchor using T4 DNA ligase. After imaging of the ligated products, the ligated anchor-probe molecules are denatured. The process of hybridization, ligation, imaging, and denaturing is repeated five times using new sets of fluorescently labeled 9-mer probes that contain known bases at the n+1, n+2, n+3, and n+4 positions.

A further sequencing platform includes nanopore sequencing (Oxford Nanopore). Nanopore detection arrays are described in US2011/0177498; US2011/0229877; US2012/0133354; WO2012/042226; WO2012/107778, and have been used for nucleic acid sequencing as described in US2012/0058468; US2012/0064599; US2012/0322679 and WO2012/164270, all of which are hereby incorporated by reference. A single molecule of DNA can be sequenced directly using a nanopore, without the need for an intervening PCR amplification step or a chemical labelling step or the need for optical instrumentation to identify the chemical label. Commercially available nanopore nucleic acid sequencing units are developed by Oxford Nanopore (Oxford, United Kingdom). The GridION™ system and miniaturised MinION™ device are designed to provide novel qualities in molecular sensing such as real-time data streaming, improved simplicity, efficiency and scalability of workflows and direct analysis of the molecule of interest. Using the Oxford Nanopore nanopore sequencing platform, an ionic current is passed through the nanopore by setting a voltage across this membrane. If an analyte passes through the pore or near its aperture, this event creates a characteristic disruption in current. Measurement of that current makes it possible to identify the molecule in question. For example, this system can be used to distinguish between the four standard DNA bases G, A, T and C, and also modified bases. It can be used to identify target proteins, small molecules, or to gain rich molecular information, for example to distinguish between the enantiomers of ibuprofen or study molecular binding dynamics. These nanopore arrays are useful for scientific applications specific for each analyte type; for example, when sequencing DNA, the technology may be used for resequencing, de novo sequencing, and epigenetics.

### III. NEXT-GENERATION SEQUENCING DATA ANALYSIS

One embodiment of an algorithm to analyze NGS reads from FASTA files starts with sequence alignment. First, C and T nucleotides from all reads and reference sequences are converted to Y to handle any degree of bisulfite conversion. The NGS reads are then aligned to the reference sequences to determine which original nucleic acid molecule and positions the NGS reads correspond to. In some embodiments, alignment is performed using software such as Bowtie 2 to account for potential sequencing errors. In some embodiments, alignment is performed using software based on an exact match, such as through hash tables, suffix trees, or the Boyer-Moore algorithm. After aligning the NGS reads to reference sequences, the conversion rate of each nucleotide position that was originally a C is calculated by dividing the number of reads with a T at that position divided by the sum of the number of reads with a T or a C at that position.

### IV. DEFINITIONS

A “target nucleic acid,” as used herein, is any nucleic acid having a known or partially known sequence for which low-yield bisulfite conversion analysis is desired. A target nucleic acid can be a single nucleic acid molecule or a plurality of nucleic acid molecules. Also, a target nucleic acid molecule can be of biological or synthetic origin. Examples of nucleic acid molecules include genomic DNA, cDNA, RNA, amplified DNA, a pre-existing nucleic acid library, etc. Nucleic acids in a nucleic acid sample being analyzed (or processed) in accordance with the present disclosure can be from any nucleic acid source. As such, nucleic acids in a nucleic acid sample can be from virtually any nucleic acid source, including but not limited to genomic DNA, complementary DNA (cDNA), RNA (e.g., messenger RNA, ribosomal RNA, short interfering RNA, microRNA, etc.), plasmid DNA, mitochondrial DNA, etc. Furthermore, as any organism can be used as a source of nucleic acids to be processed in accordance with the present disclosure, no limitation in that regard is intended. Exemplary organisms include, but are not limited to, plants, animals (e.g., reptiles, mammals, insects, worms, fish, etc.), bacteria, fungi (e.g., yeast), viruses, etc. In certain embodiments, the nucleic acids in the nucleic acid sample are derived from a mammal, where in certain embodiments the mammal is a human. A nucleic acid molecule of interest can be a single nucleic acid molecule or a plurality of nucleic acid molecules. Also, a target nucleic acid molecule can be of biological or synthetic origin. Examples of nucleic acid molecules include genomic DNA, cDNA, cell-free DNA (cfDNA), RNA, amplified DNA, a pre-existing nucleic acid library, etc. In some aspects, the target nucleic acid is a double-stranded DNA molecule, such as, for example, human genomic DNA.

“Amplification,” as used herein, refers to any in vitro process for increasing the number of copies of a nucleotide sequence or sequences. Nucleic acid amplification results in the incorporation of nucleotides into DNA or RNA. As used herein, one amplification reaction may consist of many rounds of DNA replication. For example, one PCR reaction may consist of 30-100 “cycles” of denaturation and replication.

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill in the art, e.g., exemplified by the references: McPherson et al., editors, PCR: A Practical Approach and PCR2: A Practical Approach (IRL Press, Oxford, 1991 and 1995, respectively).

“Primer” means an oligonucleotide, either natural or synthetic that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Usually primers are extended by a DNA polymerase. Primers are generally of a length compatible with its use in synthesis of primer extension products, and are usually are in the range of between 8 to 100 nucleotides in length, such as 10 to 75, 15 to 60, 15 to 40, 18 to 30, 20 to 40, 21 to 50, 22 to 45, 25 to 40, and so on, more typically in the range of between 18-40, 20-35, 21-30 nucleotides long, and any length between the stated ranges. Typical primers can be in the range of between 10-50 nucleotides long, such as 15-45, 18-40, 20-30, 21-25 and so on, and any length between the stated ranges. Primers may be no more than about 10, 12, 15, 20, 21, 22, 23, 24, 25, 26, 27, 28, 29, 30, 35, 40, 45, 50, 55, 60, 65, or 70 nucleotides in length.

The term “PCR” encompasses derivative forms of the reaction, including but not limited to, RT-PCR, real-time PCR, nested PCR, quantitative PCR, multiplexed PCR, assembly PCR and the like. Reaction volumes range from a few hundred nanoliters, e.g., 200 nL, to a few hundred microliters, e.g., 200 μL. “Reverse transcription PCR,” or “RT-PCR,” means a PCR that is preceded by a reverse transcription reaction that converts a target RNA to a complementary single stranded DNA, which is then amplified, e.g., Tecott et al., U.S. Pat. No. 5,168,038. “Real-time PCR” means a PCR for which the amount of reaction product, i.e., amplicon, is monitored as the reaction proceeds. There are many forms of real-time PCR that differ mainly in the detection chemistries used for monitoring the reaction product, e.g., Gelfand et al., U.S. Pat. No. 5,210,015 (“Taqman”); Wittwer et al., U.S. Pat. Nos. 6,174,670 and 6,569,627 (intercalating dyes); Tyagi et al., U.S. Pat. No. 5,925,517 (molecular beacons). Detection chemistries for real-time PCR are reviewed in Mackay et al., Nucleic Acids Research, 30:1292-1305 (2002). “Nested PCR” means a two-stage PCR wherein the amplicon of a first PCR becomes the sample for a second PCR using a new set of primers, at least one of which binds to an interior location of the first amplicon. As used herein, “initial primers” in reference to a nested amplification reaction mean the primers used to generate a first amplicon, and “secondary primers” mean the one or more primers used to generate a second, or nested, amplicon. “Multiplexed PCR” means a PCR wherein multiple target sequences (or a single target sequence and one or more reference sequences) are simultaneously carried out in the same reaction mixture, e.g. Bernard et al. (1999) Anal. Biochem., 273:221-228 (two-color real-time PCR). Usually, distinct sets of primers are employed for each sequence being amplified. “Quantitative PCR” means a PCR designed to measure the abundance of one or more specific target sequences in a sample or specimen. Techniques for quantitative PCR are well-known to those of ordinary skill in the art, as exemplified in the following references: Freeman et al., Biotechniques, 26:112-126 (1999); Becker-Andre et al., Nucleic Acids Research, 17:9437-9447 (1989); Zimmerman et al., Biotechniques, 21:268-279 (1996); Diviacco et al., Gene, 122:3013-3020 (1992); Becker-Andre et al., Nucleic Acids Research, 17:9437-9446 (1989); and the like.

Varied choices of polymerases exist with different properties, such as temperature, strand displacement, and proof-reading. Amplification can be isothermal, such as multiple displacement amplification (MDA) described by Dean et al., Comprehensive human genome amplification using multiple displacement amplification, Proc. Natl. Acad. Sci. U.S.A., vol. 99, p. 5261-5266. 2002; also Dean et al., Rapid amplification of plasmid and phage DNA using phi29 DNA polymerase and multiply-primed rolling circle amplification, Genome Res., vol. 11, p. 1095-1099. 2001; also Aviel-Ronen et al., Large fragment Bst DNA polymerase for whole genome amplification of DNA formalin-fixed paraffin-embedded tissues, BMC Genomics, vol. 7, p. 312. 2006. Amplification can also cycle through different temperature regiments, such as the traditional polymerase chain reaction (PCR) popularized by Mullis et al., Specific enzymatic amplification of DNA in vitro: The polymerase chain reaction. Cold Spring Harbor Symp. Quant. Biol., vole 51, p. 263-273. 1986. Other methods include Polony PCR described by Mitra and Church, In situ localized amplification and contact replication of many individual DNA molecules, Nuc. Acid. Res., vole 27, pages e34. 1999; emulsion PCR (ePCR) described by Shendure et al., Accurate multiplex polony sequencing of an evolved bacterial genome, Science, vol. 309, p. 1728-32. 2005; and Williams et al., Amplification of complex gene libraries by emulsion PCR, Nat. Methods, vol. 3, p. 545-550. 2006. Any amplification method can be combined with a reverse transcription step, a priori, to allow amplification of RNA. According to certain aspects, amplification is not absolutely required since probes, reporters and detection systems with sufficient sensitivity can be used to allow detection of a single molecule using template non-hybridizing nucleic acid structures described. Ways to adapt sensitivity in a system include choices of excitation sources (e.g. illumination) and detection (e.g. photodetector, photomultipliers). Ways to adapt signal level include probes allowing stacking of reporters, and high intensity reporters (e.g. quantum dots) can also be used.

Exemplary methods for amplifying nucleic acids include the polymerase chain reaction (PCR) (see, e.g., Mullis et al. (1986) Cold Spring Harb. Symp. Quant. Biol. 51 Pt 1:263 and Cleary et al. (2004) Nature Methods 1:241; and U.S. Pat. Nos. 4,683,195 and 4,683,202), anchor PCR, RACE PCR, ligation chain reaction (LCR) (see, e.g., Landegran et al. (1988) Science 241:1077-1080; and Nakazawa et al. (1994) Proc. Natl. Acad. Sci. U.S.A. 91:360-364), self sustained sequence replication (Guatelli et al. (1990) Proc. Natl. Acad. Sci. U.S.A. 87:1874), transcriptional amplification system (Kwoh et al. (1989) Proc. Natl. Acad. Sci. U.S.A. 86:1173), Q-Beta Replicase (Lizardi et al. (1988) BioTechnology 6:1197), recursive PCR (Jaffe et al. (2000) J. Biol. Chem. 275:2619; and Williams et al. (2002) J. Biol. Chem. 277:7790), the amplification methods described in U.S. Pat. Nos. 6,391,544, 6,365,375, 6,294,323, 6,261,797, 6,124,090 and 5,612,199, isothermal amplification (e.g., rolling circle amplification (RCA), hyperbranched rolling circle amplification (HRCA), strand displacement amplification (SDA), helicase-dependent amplification (HDA), PWGA) or any other nucleic acid amplification method using techniques well known to those of skill in the art.

As used herein in relation to a nucleotide sequence, “substantially known” refers to having sufficient sequence information in order to permit preparation of a nucleic acid molecule, including its amplification. This will typically be about 100%, although in some embodiments some portion of an adaptor sequence is random or degenerate. Thus, in specific embodiments, substantially known refers to about 50% to about 100%, about 60% to about 100%, about 70% to about 100%, about 80% to about 100%, about 90% to about 100%, about 95% to about 100%, about 97% to about 100%, about 98% to about 100%, or about 99% to about 100%.

### V. KITS

The technology herein includes kits for performing bisulfite conversion and generating sequencing libraries. A “kit” refers to a combination of physical elements. For example, a kit may include, for example, one or more components, such as specific primers, enzymes, reaction buffers, an instruction sheet, and other elements useful to practice the technology described herein. These physical elements can be arranged in any way suitable for carrying out the disclosure.

The components of the kits may be packaged either in aqueous media or in lyophilized form. The container means of the kits will generally include at least one vial, test tube, flask, bottle, syringe or other container means, into which a component may be placed, and preferably, suitably aliquoted (e.g., aliquoted into the wells of a microtiter plate). Where there is more than one component in the kit, the kit also will generally contain a second, third or other additional container into which the additional components may be separately placed. However, various combinations of components may be comprised in a single vial. The kits of the present disclosure also will typically include a means for containing the nucleic acids, and any other reagent containers in close confinement for commercial sale. Such containers may include injection or blow molded plastic containers into which the desired vials are retained.

A kit will also include instructions for employing the kit components as well the use of any other reagent not included in the kit. Instructions may include variations that can be implemented. It is contemplated that such reagents are embodiments of kits of the disclosure. Such kits, however, are not limited to the particular items identified above.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Structure Validation Via Low-Yield Bisulfite Conversion

To independently experimentally validate whether the predicted structures based on TEEM parameters are more accurate than the standard parameters, a new method for chemically probing DNA structure was developed. Bisulfite conversion is a chemical process in which unmethylated cytosine (C) nucleotides are converted into uracil (U) nucleotides, and has been typically used for studying epigenetic modification. Literature reports have suggested that strong secondary structures can adversely affect the efficiency of bisulfite conversion, so it was hypothesized that even weak secondary structures could potentially be distinguished via different bisulfite conversion efficiencies, if the reaction time and conditions were modified to reduce overall conversion yield. Use of next-generation sequencing (NGS) to analyze the converted DNA oligos allows highly precise quantitation of conversion efficiencies at single-molecule and single-nucleotide resolution (FIG. 1).

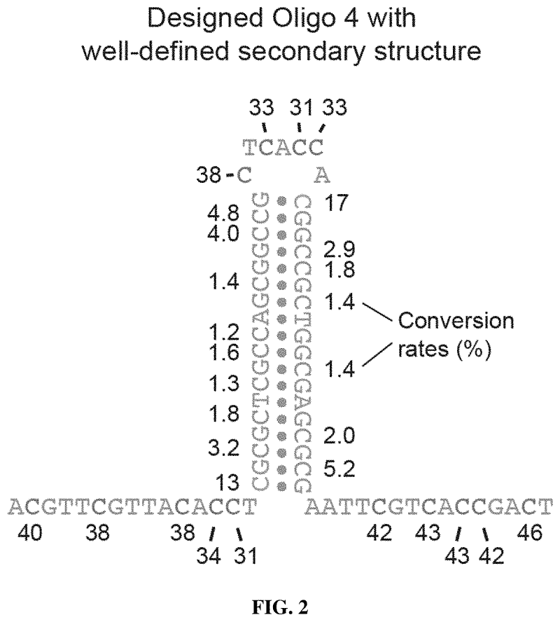

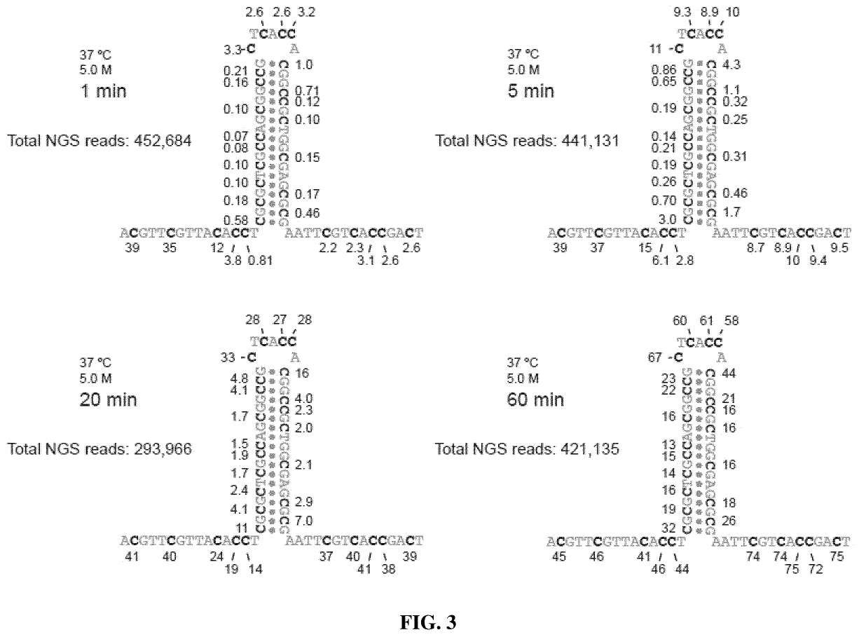

To validate the ability for low-yield bisulfite conversion to capture nucleotide base-pair states (i.e., accessibility), a control oligonucleotide (Oligo 4) that contains a single well-defined minimum free energy structure predicted identically by both the TEEM and SantaLucia thermodynamic parameters was rationally designed. Oligo 4 was subject to a 5 min bisulfite conversion reaction at 55° C., using a standard solution of 5 M sodium bisulfite. Analysis of the NGS reads for this oligo after the sodium bisulfite reaction showed that C nucleotides at different positions were converted with between 1% and 46% efficiency (based on the fraction of NGS reads showing C vs. T at the position, FIG. 2). Importantly, all 14 C nucleotides predicted to be in unpaired states showed high (>30%) conversion rates, and all 16 C nucleotides predicted to be in paired states showed low (<20%) conversion rates. This result unambiguously shows that low-yield bisulfite conversion rate is strongly correlated with the likelihood at equilibrium of finding the nucleotide in an unpaired state.

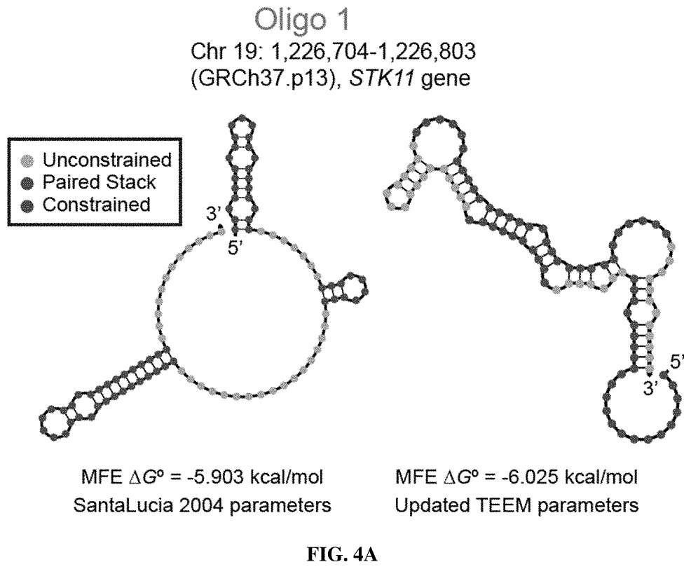

Interestingly, the closing nucleotides of the hairpin stem in Oligo 1 exhibit significantly higher conversion rates than other nucleotides in the hairpin stem, with conversion rates of 13% and 17%, compared to no higher than 5.2% for nucleotides more internal to the hairpin stem. This implies that bisulfite conversion is a dynamic process—as closing nucleotides of the hairpin stem are converted and become unpaired, the more interior bases now become closer to the ends of the stem, and more accessible to chemical reaction. Thus, a time series of the target nucleic acid subject to differing durations of bisulfite conversion can provide an additional layer of information regarding the “depth” of the structure that a nucleotide participates in. FIG. 3 supports this interpretation; in the 20-minute conversion reaction at 37° C., a graded conversion rate was observed where nucleotides deeper and deeper in to the hairpin stem have successively lower conversion rates.

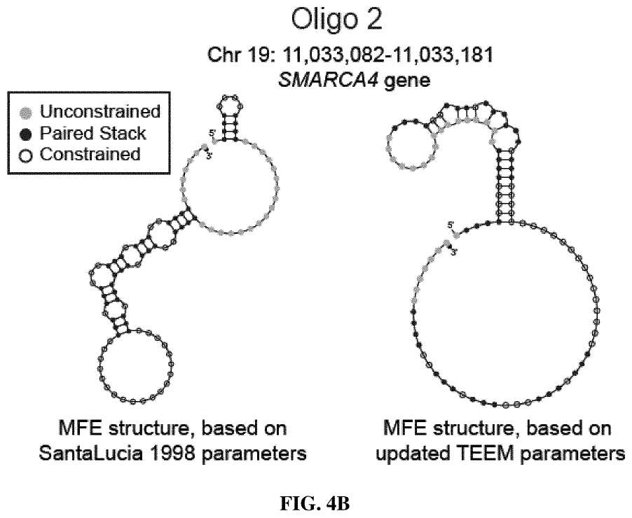

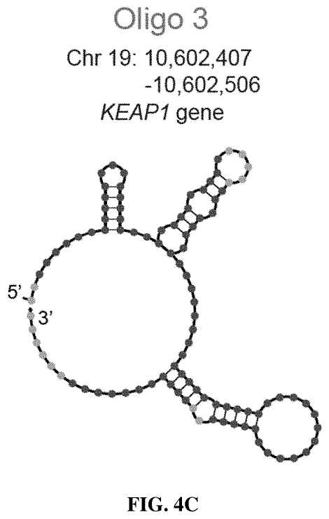

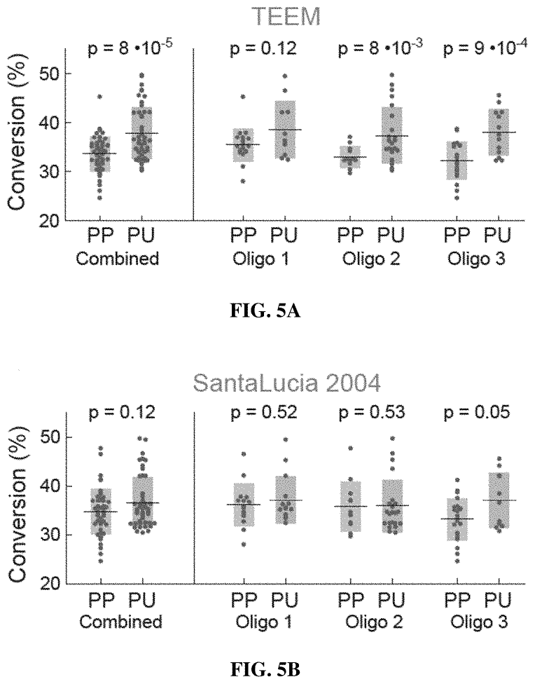

The inferred accessibility of different C nucleotides based on low-yield bisulfite conversion rates can be used to inform nucleic acid structures. In some embodiments, low-yield bisulfite conversion and NGS results can be used to favor one of multiple proposed structures for the same nucleic acid molecule. To this end, low-yield bisulfite conversion was performed on the 3 sequences described in FIGS. 4A-C. For example, FIG. 4B shows the predicted structure of a subsequence of the SMARCA4 gene, with the left structure using one set of published thermodynamics parameters, and the right structure using a different set of thermodynamics parameters. The C nucleotides in each oligo sequence were grouped into either predicted paired (PP) or predicted unpaired (PU), based on the TEEM parameters or the SantaLucia 2004 parameters. The PP nucleotides thus should exhibit lower bisulfite conversion yield than the PU nucleotides in the correct structure. Under TEEM parameters, the PP nucleotides exhibited a statistically significant lower mean conversion rate than PU nucleotides (p=8×10⁻⁵ via Mann-Whitney U test) (FIG. 5A). In contrast, under SantaLucia parameters, PP and PU nucleotides do not exhibit a statistically significant difference in the mean conversion rate (p=0.12) (FIG. 5B). Low-yield bisulfite conversion was also performed for 20 minutes at 55° C. rather than 5 minutes, and the results are similar, with TEEM parameter PP and PU nucleotides being statistically different in mean conversion rates (p=5×10⁻⁵), but not the SantaLucia ones (p=0.14). Thus, the low-yield bisulfite conversion method supports TEEM thermodynamic parameters as predicting the base pair status of nucleotides in an oligo more accurately than literature parameters.

The quantitative difference between conversion rates for PP and PU nucleotides (TEEM parameters) is smaller than the difference in conversion rates for Oligo 4 (FIG. 2). This is not unexpected, as the duplex stems in the MFE structures for Oligos 1, 2, and 3 are significantly shorter than for Oligo 4. Base pairs near the ends of duplex stems are prone to base breathing, and thus spend a greater fraction of the time in a single-stranded state, resulting in higher bisulfite conversion rates. For example, the two C nucleotides that were closing base pairs of the duplex stem in Oligo 4 exhibited conversion yields of 13% and 17%, compared to all other paired C nucleotides with less than 6% conversion rate.

The TEEM method for thermodynamic characterization of DNA motifs is significantly higher throughput than all previous methods, and simultaneously is more precise (Table 1). These improved capabilities allow for the accurate measurement of the ΔΔG° of motifs, such as bulges and mismatch bubbles, across a wide temperature range, which lead to the realization that previously reported ΔH° and ΔS° were inaccurately extrapolated from DNA melt experiments. These studies uncovered a new molecular biophysical phenomenon: that almost all destabilizing DNA motifs are driven primarily by enthalpy. Further theoretical studies and molecular dynamics modeling is needed to understand the fundamental biophysics of DNA folding and hybridization.

TABLE 1
Comparison of DNA thermodynamics measurement methods

| | Throughput | ΔG° | | | Valid | |
|---|---|---|---|---|---|---|
| | (# of | standard | DNA | | Temp. | |
| Method | ΔG° values) | error | conc. | | Range | |
| UV Melt Analysis | 31/plate | 0.4 kcal/mol | 1 | μM | 5° | C. |
| High Resolution | 31/plate | 1.0 kcal/mol | 0.2 | μM | 5° | C. |
| Melt | | | | | | |
| Isothermal | 1/cell | 0.1 kcal/mol | 5000 | μM | 1° | C. |
| Titration Calo- | | | | | | |
| rimetry (ITC) | | | | | | |
| Differential | 1/cell | 0.1 kcal/mol | 100 | μM | 5° | C. |
| Scanning Calo- | | | | | | |
| rimetry (DSC) | | | | | | |
| TEEM | 1200/plate | 0.05 kcal/mol | 1 | μM | 40+° | C. |

It is surprising that DNA thermodynamics, which have been characterized systematically in many studies starting 35 years ago, can have glaring inaccuracies and undiscovered phenomenon. To support the validity of these conclusions, this new method was rigorously verified via a series of measurements of the same DNA motif in different contexts, which were systematically replicated to minimize statistical error (at least 9 replicate experiments for each motif). Based on these results, the phenomenon observed is indeed reflective of natural biophysics.

The accuracy of TEEM results relies upon the assumption that the motif ΔΔG° can be approximated as the difference of reaction ΔG° values, which holds true when the single-stranded reference and variant DNA sequences exhibit similar folding energies. Insofar as DNA folding software rely on imperfect thermodynamic parameters, the measured ΔΔG° values for any particular set of sequences may differ from objectively true values by a small amount. However, this systematic bias should not be temperature dependent, so the conclusion regarding the temperature invariance of ΔΔG° for DNA destabilization motifs should hold. Furthermore, these experiments on several independent DNA sequences with the same motif showed variation of less than 0.1 kcal/mol, indicating that any error is likely small.

Using the TEEM characterized thermodynamic parameters for bulges and mismatches in DNA folding software resulted in different predicted structures and/or energies in 60% of biological sequences analyzed at 55° C. Furthermore, a consistent bias to more predicted structure at lower temperatures and less predicted structure at higher temperatures was observed. The NUPACK parameter files have been made publicly available so that the updated software can be used in guiding DNA probe and primer design.

Provided herein are methods for probing with high resolution the base pairing state of each cytosine in an oligonucleotide, using low-yield bisulfite conversion and downstream NGS. In addition to independently validating the correctness of TEEM thermodynamics parameters, it is envisioned that low-yield bisulfite conversion can be a powerful new tool for analyzing the secondary and tertiary structure of nucleic acids. Compared to similar chemical probing methods such as SHAPE-Seq (Lucks et al., 2011), the present method provides higher information throughput, because each NGS read simultaneously reports on the base pair status of multiple nucleotides. On the other hand, the present methods are limited in being only capable of probing C nucleotides, and thus may be unsuitable for A/T-rich sequences.

Although the TEEM thermodynamics parameters result in improved DNA secondary structure prediction over published parameters, the imperfect anti-correlation between predicted base pair probability and bisulfite conversion rate suggests that there remain many inaccuracies in the thermodynamics parameters even with the TEEM parameter updates. For example, there are very likely still inaccuracies in the thermodynamics of hairpin loops and multiloops. Adaptation and optimization of the TEEM method may allow a systematic and comprehensive profiling of these other hybridization motifs, which could lead to even better secondary structure prediction.

### Example 2—Oligo Sequences and Concentrations Used

Table 2 lists the sequences of the DNA oligonucleotides used for demonstration of low-yield bisulfite conversion. R nucleotides indicate that the oligo was synthesized as a degenerate randomer sequence with roughly equal probability of each nucleotide being A or G at the R position.

TABLE 2
Sequences of DNA oligonucleotides

| Name | Sequence | Conc. |
|---|---|---|
| Hairpin oligo | Phosphate- | 6.7 nM |
| (Oligo 1) | CGCCTGGATGCCACAGCCAGCCGTGAGCATAGCCCGCGCTAGTCA | |
| | GTCATGGTGACCGTCACGTGGCTGCGCGTGGTTGCCATGTGGCCT | |
| | TTGGGTGGCT (SEQ ID NO: 1) | |
| Oligo 2 | Phosphate- | 6.7 nM |
| | GGCCGAGGGTGGCACGCACAGCACACCTCTCCAGCTAGTGTCAGA | |
| | GGCCACCTTCCCTTTTATGACCTCCTGGGCTCCTTTGGGACTGAC | |
| | TGGCACCTCT (SEQ ID NO: 2) | |
| 5-Adapter | ACACTCTTTCCCTACACGACGCTCTTCCGATCT | 40 nM |
| | (SEQ ID NO: 3) | |
| 3-Adapter | Phosphate-AGATCGGAAGAGCACACGTCTGAACTCCAGTC- | 40 nM |
| | C3spacer (SEQ ID NO: 4) | |
| 5-Splint | ARRTRTAACRAACRTAGATCGGAAGAGCGT (SEQ ID NO: 5) | 120 nM |
| Oligo1 | | |
| 5-Splint | RTRCCACCCTCRRCCAGATCGGAAGAGCGT (SEQ ID NO: 6) | 102 nM |
| Oligo2 | | |
| 3-Splint | GTGCTCTTCCGATCTARTCRRTRACRAATT (SEQ ID NO: 7) | 60 nM |
| Oligo1 | | |
| 3-Splint | GTGCTCTTCCGATCTARARRTRCCARTCAR (SEQ ID NO: 8) | 120 nM |
| Oligo2 | | |

### Example 3—Next-Generation Sequencing (NGS) Data Summary

Tables 3-4 summarize the number of reads that are C vs. T at each position for each oligo tested. The position of the nucleotide indicates where the C is in the hairpin oligo, counting from the 5′-end. C indicates that bisulfite conversion did not occur, and T indicates that bisulfite conversion did occur.

TABLE 3
NGS on hairpin oligo (Oligo 4)*

| | Position of nucleotide | | | | | | |
|---|---|---|---|---|---|---|---|
| | 2 | 6 | 11 | 13 | 14 | 16 | 18 |
| C | 462,288 | 482,716 | 478,274 | 510,533 | 533,688 | 675,282 | 748,601 |
| T | 310,770 | 290,342 | 294,784 | 262,525 | 239,370 | 97,776 | 24,457 |
| | Position of nucleotide | | | | | | |
| | 20 | 22 | 24 | 25 | 28 | 31 | 32 |
| C | 758,972 | 762,819 | 761,009 | 764,000 | 762,477 | 742,296 | 736,272 |
| T | 14,086 | 10,239 | 12,049 | 9,058 | 10,581 | 30,762 | 36,786 |
| | Position of nucleotide | | | | | | |
| | 34 | 36 | 38 | 39 | 41 | 44 | 45 |
| C | 477,699 | 520,677 | 534,556 | 519,445 | 641,325 | 750,402 | 758,908 |
| T | 295,359 | 252,381 | 238,502 | 253,613 | 131,733 | 22,656 | 14,150 |
| | Position of nucleotide | | | | | | |
| | 47 | 51 | 55 | 57 | 63 | 66 | 68 |
| C | 762,155 | 761,999 | 757,764 | 732,983 | 446,582 | 443,708 | 443,706 |
| T | 10,903 | 11,059 | 15,294 | 40,075 | 326,476 | 329,350 | 329,352 |

| | Position of nucleotide | |
|---|---|---|
| | 69 | 72 |
| C | 447,470 | 416,863 |
| T | 325,588 | 356,195 |

*5 min bisulfite conversion, n = 773,058 NGS reads

TABLE 4
NGS on Oligo 2*

| | Position of nucleotide | | | | | | |
|---|---|---|---|---|---|---|---|
| | 3 | 4 | 13 | 15 | 17 | 19 | 22 |
| C | 69,673 | 74,903 | 74,573 | 74,762 | 75,553 | 74,588 | 76,850 |
| T | 40,830 | 35,600 | 35,930 | 35,741 | 34,950 | 35,915 | 33,653 |
| | Position of nucleotide | | | | | | |
| | 24 | 26 | 27 | 29 | 31 | 32 | 35 |
| C | 75,668 | 74,720 | 76,596 | 72,436 | 72,935 | 77,089 | 70,819 |
| T | 34,835 | 35,783 | 33,907 | 38,067 | 37,568 | 33,414 | 39,684 |
| | Position of nucleotide | | | | | | |
| | 42 | 48 | 49 | 51 | 52 | 55 | 56 |
| C | 72,222 | 72,733 | 77,677 | 74,806 | 74,748 | 69,530 | 71,024 |
| T | 38,281 | 37,770 | 32,826 | 35,697 | 35,755 | 40,973 | 39,479 |
| | Position of nucleotide | | | | | | |
| | 57 | 66 | 67 | 69 | 70 | 75 | 77 |
| C | 72,308 | 70,361 | 72,168 | 70,380 | 72,633 | 67,973 | 69,493 |
| T | 38,195 | 40,142 | 38,335 | 40,123 | 37,870 | 42,530 | 41,010 |
| | Position of nucleotide | | | | | | |
| | 78 | 86 | 90 | 94 | 96 | 97 | 99 |
| C | 72,191 | 64,776 | 57,787 | 55,588 | 60,377 | 62,406 | 58,941 |
| T | 38,312 | 45,727 | 52,716 | 54,915 | 50,126 | 48,097 | 51,562 |

*5 min bisulfite conversion, n = 110,503 NGS reads

### Example 4—Population Analysis Via Low-Yield Bisulfite Conversion

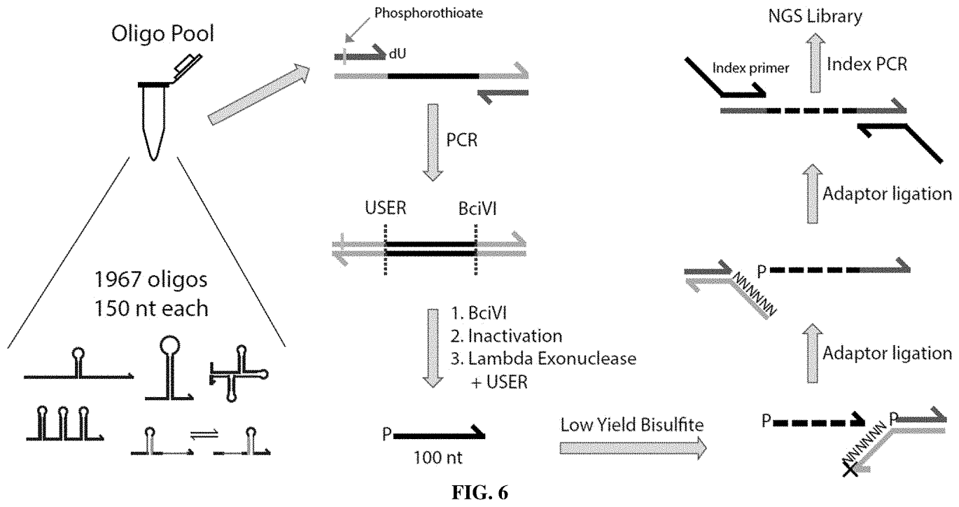

Starting from a pool of 1,967 oligonucleotides (SEQ ID NOS: 10-1978), which were all 150 nucleotides long, PCR was performing with one of the primers having a dU at its 3′ end and three phosphorothioate bonds at its 5′ end (see FIG. 6). The resulting amplicons had both USER and BciVI restriction sites. After using BciVI, Lambda exonuclease, and USER, primer binding regions were removed leaving only the 100 nucleotide-long area of interest. Low-yield bisulfite conversion was then performed, followed by two ligation steps to attach NGS adaptors with the help of random hexamers on the guiding oligos. NGS library preparation was finished by running index PCR.

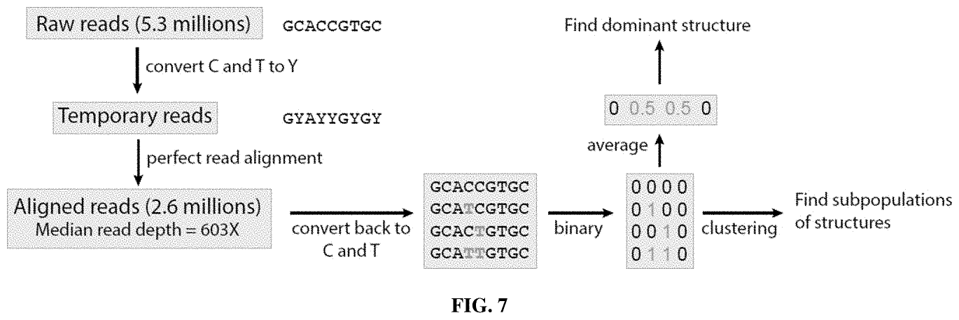

An example of the data processing methods is shown in FIG. 7. As shown, the NGS library generated 5.3 million raw reads with some of the C converted to T as a result of low-yield bisulfite conversion. For alignment purpose, all C and T were written as Y temporarily in both the raw reads and the reference sequences. After selecting only perfectly aligned reads, 2.6 million reads were left with the median read depth of 603x. All C and T were then recovered from Y. To make data processing faster and more efficient, C converted to T during low-yield bisulfite conversion was treated as 1 whereas C unconverted to T was treated as 0. Finding the dominant structure of an oligo was done by using average conversion of each C, and finding subpopulations of structures for each oligo was done by clustering the reads instead of calculating the average conversion.

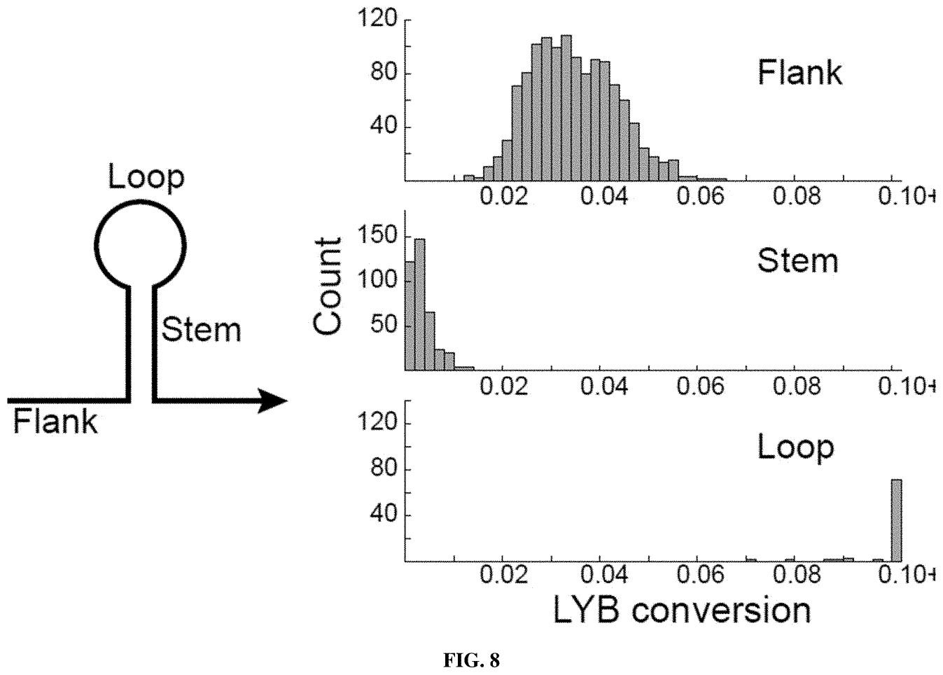

Motifs of a secondary structure were classified into three groups: Flank, Stem, and Loop. When mean conversions of each C were grouped according to where the C is located, there were clear differences among three groups (FIG. 8). Conversions at Stem were much lower than those at Flank because Cs in the Stem are paired and less exposed to bisulfite solution. On the other hand, conversions at Loop were much higher, which makes it possible to distinguish loop structures from other unpaired and open structures.

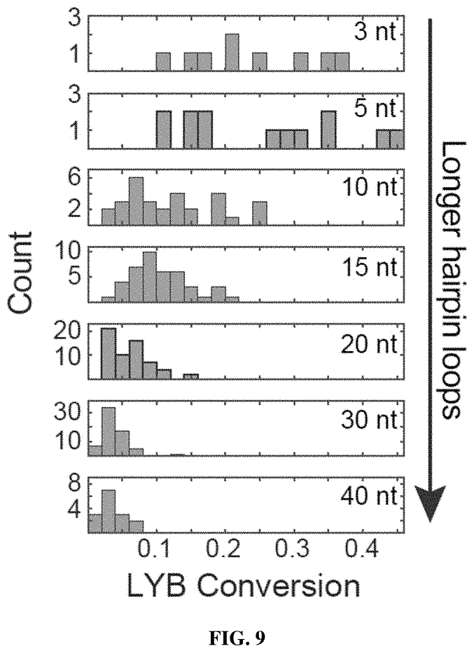

Mean conversions at Loop were grouped by the length of Loop when the length of Stem was always 20 nucleotides. Conversion at Loop were affected by the length of the loop (FIG. 9). As the length of a loop gets longer, the characteristic high conversions gradually disappear. Conversions at Loop longer than 20 nucleotides did not show any significant difference from conversions at Flank.

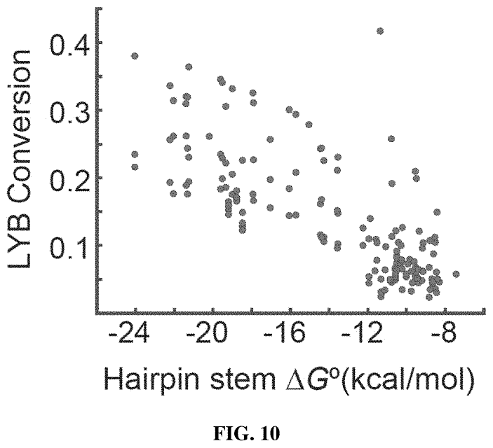

In addition, mean conversion at Loop were plotted against the free energy of hybridization of their own Stem. Stronger Stems showed higher conversions in general (FIG. 10), and this conversion pattern can be used to predict a structure of an oligonucleotide.

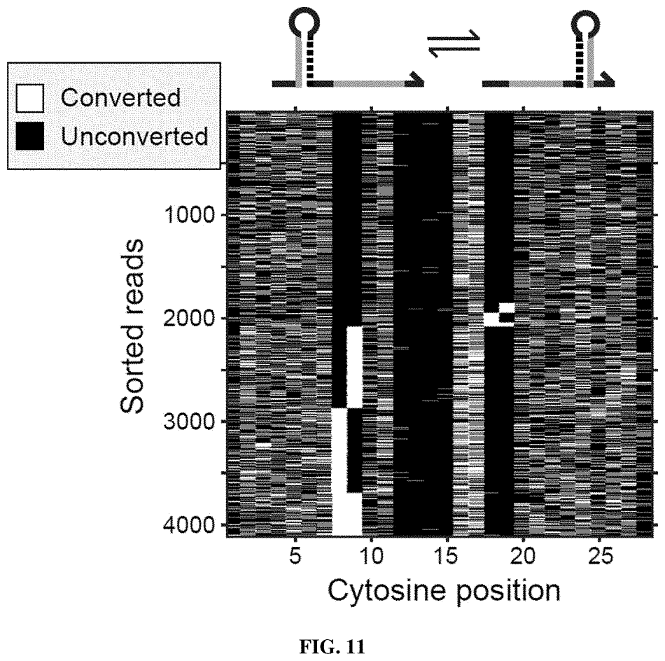

Conversions of an oligonucleotide that was designed to have two competing structures in equilibrium are shown in FIG. 11. All reads of the oligonucleotide were visualized by displaying converted C in white and unconverted C in black. Each row shows all C in one read, and each column shows specific positions of C. When the reads were sorted by conversions at the 8^{th} and 9^{th} Cs, it became clear that conversions at the 18^{th} and 19^{th} Cs did not happen when either the 8^{th} or 9^{th} C were converted. Because the 8^{th} and 9^{th} Cs had to compete with 18^{th} and 19^{th} Cs to form a hairpin stem structure, they were mutually exclusive. Black columns in the middle shows the Cs that were on Stem in both structures.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Lucks et al., “Multiplexed RNA structure characterization with selective 2′-hydroxyl acylation analyzed by primer extension sequencing (SHAPE-Seq),” Proc. Natl. Acad. Sci. U.S.A., 108:11063-11068, 2011.

1. A method for low-yield conversion of unmethylated cytosine nucleotides to uracil (U) nucleotides in a target nucleic acid molecule, the method comprising:
(a) introducing a bisulfite solution to a sample comprising the target nucleic acid molecule to achieve a final bisulfite concentration of between 0.1 M and 10 M;
(b) allowing the bisulfite conversion to react a temperature between 4° C. and 70° C.;
(c) stopping the bisulfite conversion reaction through removal of the excess bisulfite; and
(d) performing desulfonation.

2. The method of claim 1, wherein unmethylated cytosines that do not participate in base pairing have a C to U conversion rate of no more than 90%.

3. The method of claim 1, wherein the bisulfite concentration is between 4 M and 10 M, and the reaction proceeds at a temperature between 4° C. and 55° C. for between 1 minute and 60 minutes.

4. The method of claim 1, wherein the bisulfite concentration is between 4 M and 10 M, and the reaction proceeds at a temperature between 45° C. and 70° C. for between 10 seconds and 20 minutes.

5. The method of claim 1, wherein the bisulfite concentration is between 0.5 M and 5 M, and the reaction proceeds at a temperature between 45° C. and 70° C. for between 5 minutes and 60 minutes.

6. The method of claim 1, wherein the bisulfite concentration is between 0.1 M and 1 M, and the reaction proceeds at a temperature between 37° C. and 70° C. for between 30 minutes and 12 hours.

7. The method of claim 1, wherein the target nucleic acid molecule is DNA.

8. The method of claim 1, wherein the target nucleic acid molecule is RNA.

9. The method of claim 1, wherein the bisulfite conversion is stopped by separating the bisulfite from the target nucleic acid.

10. (canceled)

11. The method of claim 1, wherein desulfonation is performed at a pH over 12.

12. A method of determining a nucleotide accessibility status of cytosine (C) nucleotides in a target nucleic acid molecule, the method comprising:
(a) performing a low-yield bisulfite conversion reaction to convert a fraction of cytosine nucleotides in a population of the target nucleic acid molecules to uracil (U) nucleotides;
(b) analyzing the converted nucleic acid molecules at each nucleotide position originally known to be a cytosine, to determine or estimate a conversion fraction of all molecules of the population that is converted at each nucleotide position originally known to be a cytosine; and
(c) mapping the conversion fraction to a nucleotide accessibility status.

13. The method of claim 12, wherein analyzing comprises sequencing the converted nucleic acid molecules.

14-15. (canceled)

16. The method of claim 13, wherein the fraction of all molecules of a target nucleic acid that are read as thymine (T) or uracil (U) at a given nucleotide is computed or estimated from sequencing data.

17-18. (canceled)

19. The method of claim 12, wherein mapping the conversion fraction to nucleotide accessibility status comprises comparing the conversion fraction at a C nucleotide position to conversion fractions of all other C nucleotide positions in the same nucleic acid species.

20. The method of claim 12, wherein mapping the conversion fraction to nucleotide accessibility status comprises comparing the conversion fraction at a C nucleotide position to conversion fractions of all other C nucleotide positions in the other all nucleic acid species within the same multiplexed conversion reaction.

21. The method of claim 12, wherein mapping the conversion fraction to nucleotide accessibility status comprises comparing the conversion fraction at a C nucleotide position to an information database of conversion fractions of C nucleotides in different accessibility states, under the same low-yield bisulfite conversion reaction conditions.

22-25. (canceled)

26. A method of determining a nucleotide accessibility status of cytosine (C) nucleotides in a target nucleic acid molecule, the method comprising:
(a) performing a low-yield bisulfite conversion reaction to convert a fraction of cytosine nucleotides in a first sample of a population of the target nucleic acid molecules to uracil (U) nucleotides for a defined amount of time t₁;
(b) constructing a next-generation sequencing (NGS) library based on the converted nucleic acid molecules and running NGS;
(c) analyzing the NGS reads to determine, for each nucleotide position originally known to be a cytosine, the fraction of all molecules of the converted nucleic acid molecules that is converted to determine a bisulfite conversion rate;
(d) repeating steps (a) through (c) for a second sample of the same population of nucleic acid molecules, subject to a low-yield bisulfite conversion reaction for a different amount of time t₂; and
(e) determining the accessibility of a nucleotide in the target nucleic acid molecules based on the combined analysis of the bisulfite conversion rates from both time points.

27-28. (canceled)

29. The method of claim 26, wherein mapping the conversion fraction to nucleotide accessibility status comprises comparing the conversion fraction at a C nucleotide position to conversion fractions of all other C nucleotide positions in the same nucleic acid species.

30. The method of claim 26, wherein mapping the conversion fraction to nucleotide accessibility status comprises comparing the conversion fraction at a C nucleotide position to conversion fractions of all other C nucleotide positions in the other all nucleic acid species within the same multiplexed conversion reaction.

31. The method of claim 26, wherein mapping the conversion fraction to nucleotide accessibility status comprises comparing the conversion fraction at a C nucleotide position to an information database of conversion fractions of C nucleotides in different accessibility states, under the same low-yield bisulfite conversion reaction conditions.

32-35. (canceled)
